(19) United States
(12) Patent Application Publication
(10) Pub. No.: US 2021/0393821 A1
(43) Pub. Date: Dec. 23, 2021

(54) GAMING CHIP DISINFECTION

(71) Applicant: CleanLight Solutions LLC, Woodland Park, CO (US)

(72) Inventors: Robert Dean McCoy, Woodland Park, CO (US); Denise G. McCoy, Woodland Park, CO (US); Barry E. Caldwell, Colorado Springs, CO (US)

(21) Appl. No.: 17/350,454

(22) Filed: Jun. 17, 2021

Related U.S. Application Data
(60) Provisional application No. 63/042,194, filed on Jun. 22, 2020.

(51) Int. Cl. A61L 2/10; A61L 2/20; G07F 17/32

(52) U.S. Cl. CPC A61L 2/10; G07F 17/3241; G07F 17/3202; A61L 2/20

(57) ABSTRACT

An apparatus for disinfecting gaming chips includes an ingress, a germicidal radiation source, an egress, an interior, a security system, and a controller. The apparatus is configured to control the passing of untreated gaming chips from the ingress to the interior of the apparatus, then to the egress. The interior of the apparatus has at least one germicidal radiation source. The germicidal radiation source treats the gaming chips such that the gaming chips at the egress are disinfected. The security system is configured to restrict user access to the interior, ingress, and/or egress of the apparatus. The controller is operably coupled with the security system, ingress, egress, and the interior of the apparatus. A method for disinfecting gaming chips includes passing of contaminated gaming chips from an ingress through a plurality of passages in direct line of sight to a radiation sources, irradiating the chips, then to an egress.

### CROSS REFERENCE TO RELATED APPLICATIONS

This application claims the priority benefit of U.S. Provisional Application No. 63/042,194 filed Jun. 22, 2020, pending, which is incorporated herein by reference in its entirety.

### TECHNICAL FIELD

The present disclosure relates to gaming chip disinfecting apparatus and methods for disinfecting gaming chips.

### BACKGROUND

In casinos and other gaming venues, gaming chips are likely to be handled by many different individuals. A single gaming chip may move between a dealer and multiple players during the course of play at a single game site within a gaming venue. That gaming chip may also move between gaming venue game sites, and subsequently be passed among a new set of individuals, as individual players move from one game site to another. Other individuals can also come into contact with that gaming chip during the course of normal gaming venue operations, such as when a casino cashier exchanges gaming chips for currency, or when game sites are stocked with additional gaming chips.

It has been shown that bacterial and viral microorganisms are able to survive on inert surfaces for extended periods of time. The free movement of gaming chips between persons in a gaming venue provides an opportunity for bacterial and viral microorganisms to be passed between the various individuals handling those gaming chips thereby increasing the potential for passing infectious diseases between those persons.

### SUMMARY

The disclosure describes a gaming chip disinfection apparatus. The gaming chip apparatus includes a gaming chip ingress, a gaming chip egress, one or more germicidal radiation sources, a plurality of channels and a security system. The gaming chip ingress is arranged to control passing of contaminated gaming chips to an interior of the apparatus. The gaming chip egress is arranged to control passing of disinfected gaming chips to an exterior of the apparatus from the interior of the apparatus. The one or more germicidal radiation sources are provided at the interior of the apparatus. The channels are provided at the interior of the apparatus between the ingress and the egress and are configured to transport gaming chips from the ingress, through the disinfection apparatus and to the gaming chip egress in a controlled manner such that all surfaces of transported gaming chips are simultaneously exposed to at least one of the one or more germicidal radiation sources directly, by line-of-sight for a prescribed duration. The security system is arranged to restrict access to gaming chips at the ingress, at the egress, at the interior or a combination of these.

Another gaming chip disinfection apparatus includes a gaming chip ingress, a gaming chip egress, a plurality of passages, a plurality of germicidal radiation sources and a security feature. The gaming chip ingress is arranged to direct untreated gaming chips to an interior of the apparatus. The gaming chip egress is arranged to direct treated gaming chips to an exterior of the apparatus from the interior of the apparatus. The passages are provided at the interior of the apparatus, between the ingress and the egress and are arranged to guide gaming chips from the ingress, through the disinfection apparatus and to the gaming chip egress. The germicidal radiation sources are provided to the passages and configured to irradiate top and bottom base surfaces of the guided gaming chips concurrently. The security feature is arranged to restrict access to gaming chips at the ingress, at the egress, at the interior or a combination of these.

The disclosure also describes a method for gaming chip disinfection. The method includes supplying one or more untreated gaming chips to a gaming chip disinfection apparatus ingress arranged to control passing of untreated gaming chips to an interior of the apparatus. With a plurality of germicidal radiation sources of the gaming chip apparatus, top and bottom base surfaces of the guided gaming chips are concurrently, directly irradiated. With a plurality of passages, the gaming chips are guided from the ingress, through the disinfection apparatus and to a gaming chip egress.

### BRIEF DESCRIPTION OF THE FIGURES

The summary above, as well as the following detailed description of illustrative embodiments, is better understood when read in conjunction with the appended drawings. For the purpose of illustrating the disclosure, example constructions are shown in the drawings. However, the disclosure is not limited to specific methods and instrumentalities disclosed herein. Moreover, those having ordinary skill in the art will understand that the drawings are not to scale. Wherever possible, like elements have been indicated by identical numbers.

Embodiments of the disclosure will now be described, by way of example only, with reference to the following diagrams wherein:

FIG. 1 illustrates a side sectional view of an example gaming chip disinfection apparatus.

FIG. 2 illustrates a detail sectional view of an example passage of the gaming chip disinfection apparatus of FIG. 1.

FIG. 3 illustrates a side sectional view of an example passage or channel of the gaming chip disinfection apparatus of FIGS. 1 & 2.

FIG. 4 illustrates a side sectional view of another example gaming chip disinfection apparatus.

FIG. 5 illustrates a detail sectional view of example passages of the gaming chip disinfection apparatus of FIG. 4.

FIG. 6 illustrates a side sectional view of example passages or channels of the gaming chip disinfection apparatus of FIGS. 4 & 5.

FIG. 7 schematically illustrates a control structure in accordance with an embodiment of the disclosure.

### DETAILED DESCRIPTION

The following detailed description illustrates embodiments of the disclosure and manners by which they can be implemented. Although the best mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

Gaming chips that are disinfected against bacterial and viral microorganisms using a wet washing process cannot be immediately used. They must be dried before they can be made available for use in the gaming venue. Additionally, automated gaming chip washing methods tend to be more efficient when applied to large quantities of gaming chips at a single time. These traits make the gaming chip washing method infeasible for use in an active gaming environment where a preferably automated and relatively quick method of effectively disinfecting casino chips on a frequent basis is required.

Previously proposed Ultraviolet Germicidal Irradiation (UVGI) devices subject gaming chips to a short-wavelength ultraviolet light environment while being held stationary and perpendicular to each other in a chip rack. UVGI uses short wavelength ultraviolet light to kill or inactivate microorganisms by damaging or destroying nucleic acids and disrupting their DNA or RNA, leaving them unable to perform vital molecular biologic functions. UVGI has been shown to be an effective method of killing bacterial and viral microorganisms on surfaces such as gaming chips. However, devices with a disinfecting environment including obstacles that obstruct direct line-of-sight exposure of surfaces of the gaming chips to UVGI are unable to provide a disinfection process as effective as devices that provide direct line-of-sight exposure of all gaming chip surfaces to germicidal radiation.

Incorporating a set of dual conveying systems enables initially exposing one side of a gaming chip placed on a first conveyor to ultraviolet light, depositing onto a second conveyor and exposing the opposite side of the gaming chip to ultraviolet light. The vertical length of a device needed to realize this method makes it difficult to use at existing game sites, such as on card game tables, craps tables, or roulette tables, where such a device would need to be located to allow for the constant disinfecting of gaming chips.

Embodiments of the disclosure substantially eliminate, or at least partially address, problems in the prior art, enabling secure, dry disinfection of contaminated or untreated gaming chips quickly in a small space.

Additional aspects, advantages, features, and objects of the disclosure will be made apparent from the drawings and the detailed description of the illustrative embodiments construed in conjunction with the appended claims that follow. It will be appreciated that described features are susceptible to being combined in various combinations without departing from the scope of the disclosure as defined by the appended claims.

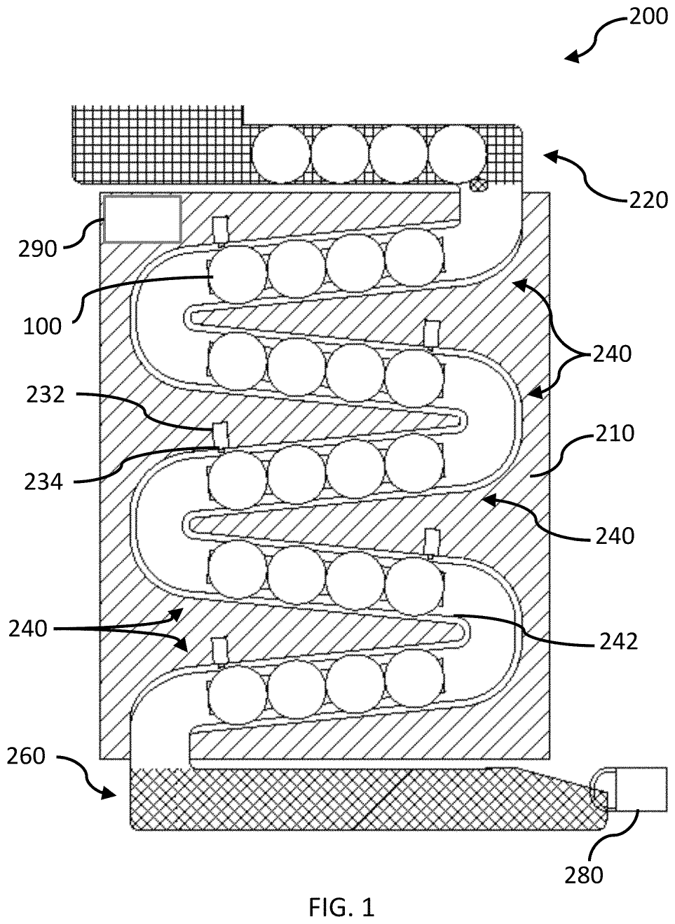

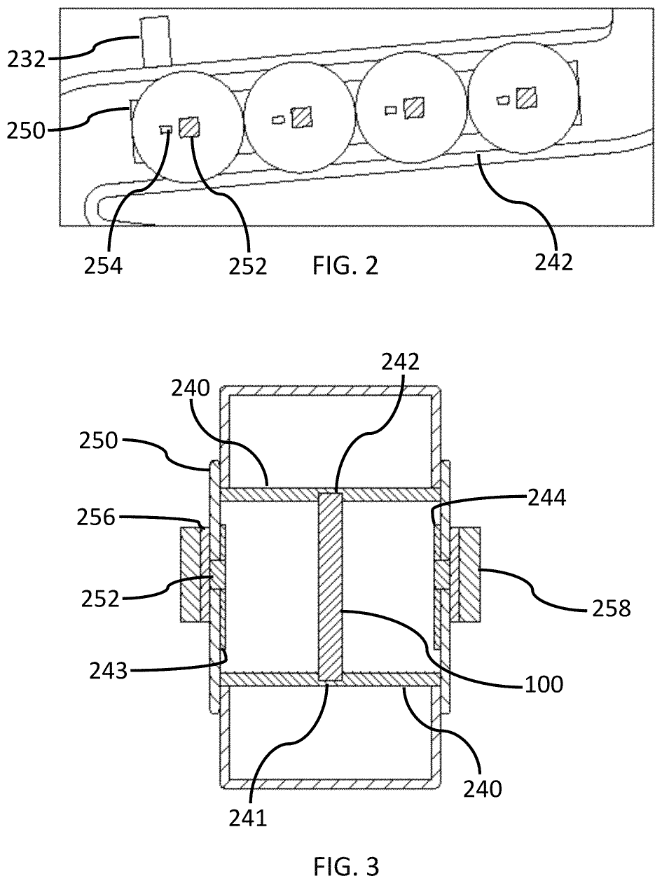

FIGS. 1-3 illustrate an example gaming chip disinfection apparatus 200 configured to disinfect gaming chips 100. Gaming chips 100 may include, but are not limited to, casino chips, gaming tokens, checks, cheques, backgammon checkers or men, checker pieces or draughts. In some cases, gaming chips are discs that are used in lieu of currency by clientele of casinos and other gaming venues to represent wagers in currency-based gambling games. In an example, gaming chips 100 may be considered to have a disk shape with a top base surface, a bottom base surface, and an edge delineating the top and bottom surfaces. In a further example, the edge has a surface area and may be considered as a third surface or a side, between the top and bottom surfaces. Gaming chips 100 can be contaminated when handled by a person, and/or through routine use of gaming chips during gaming activities, such as, but not limited to, poker, blackjack, roulette, or other betting and gambling games.

Gaming chip disinfection apparatus 200, includes a gaming chip ingress 220, a gaming chip egress 260 and one or more germicidal radiation sources 252 provided to the apparatus interior. Germicidal radiation sources 252 exhibit one or more properties that kill or inactivate microorganisms by destroying, damaging, or disrupting their nucleic acids (DNA and/or RNA) at a viral or cellular level, cell membrane, lipid and protein shell, or other critical structure; leaving them unable to perform vital cellular or viral functions which cause the organism to become non-pathogenic. Radiation sources 252 may employ germicidal electromagnetic irradiation (GI) such as ultra-violet (UV) and/or infrared (IR) electromagnetic radiation to subject gaming chips 100 to a short-wavelength (200-280 nm), ultraviolet (UV-C) light environment and/or a long-wavelength (1000-100000 nm) infrared light environment.

Throughout the interior of apparatus 200 gaming chips 100 can be guided by channels or passages 240. As used herein, the “interior” of the apparatus means, the portion of apparatus 200 separating ingress 220 and egress 260. Housing 210, which may have several exterior walls, surrounds and partially defines the interior. Apparatus 200 is configured to disinfect multiple gaming chips 100, at once, in a batch-type, semi-batch, or continuous process.

Passages 240 are configured to guide gaming chips 100 in series (FIG. 1) or along a single path from ingress 220 to egress 260. Passages 240 can include opposing walls 243 and 244 which may parallel one or more of exterior walls of housing 210. The opposing walls can be aligned substantially parallel to top and bottom surfaces of a gaming chip 100, as shown in FIG. 3. In some embodiments germicidal radiation sources 252 are provided to opposing walls 243, 244 of passages 240. In some embodiments, opposing walls 243, 244 include one or more reflective surfaces. The reflective surfaces can be configured to reflect light, and/or electromagnetic radiation emitted from radiation sources 252. While the reflective surfaces may be formed from any of a variety of materials, in an example, the reflective services include polished aluminum.

In an example, opposing walls 243, 244 may include one or more heat sink 256 configured to thermally couple with LED board assembly 258 to remove unwanted heat.

Passages 240 can be configured to transport gaming chips 100 from ingress 220 to the interior of apparatus 200 where the chips are disinfected, or irradiated, via radiation sources 252, to an egress 260 that controls the passage of disinfected gaming chips to the exterior of the apparatus. Passages 240 are configured to transport gaming chips 100 near germicidal radiation sources 252 such that gaming chips 100 are exposed to incident germicidal radiation for a length of time sufficient to disinfect them.

Support surfaces of passages 240 include grooves 241 and 242 configured to receive edges of gaming chips 100 and guide gaming chips 100 along passages 240 in an orientation suitable for irradiating at least the top and the bottom base surfaces of the gaming chips substantially concurrently, for example, such that a duration of irradiation of the top base surface overlaps, mostly or at least partially, with the duration of irradiation of the bottom base surface.

Passages 240 may be configured to guide gaming chips from ingress 220 through disinfection apparatus 200 to positions substantially aligned with radiation sources 252 and on to egress 260 under gravity feed. For example, support surfaces of passages 240 may include downward slopes or declined planes forming oblique angles with exterior walls of the housing 210 (FIGS. 1 & 2).

As used herein the term “treatment” can refer to the disinfection of contaminated gaming chips, and the term “untreated” can refer to contaminated gaming chips that have not been disinfected, irradiated, or decontaminated. In some cases, the passages are arranged inside the interior of the apparatus to guide gaming chips from the ingress, through the interior of the apparatus, to the egress.

Germicidal radiation source 252 can be an element configured to emit photons incident to the surfaces of a gaming chip and capable of disinfecting, or killing germs, such as bacteria, viruses, protozoa, or other microscopic pathogens that can live on the surface of a gaming chip. In some cases, a germicidal radiation source 252 can emit ultraviolet and/or infrared wavelength electromagnetic radiation.

As shown in FIG. 3 germicidal radiation sources 252 may be provided to passages 240 and configured to directly irradiate top and bottom base surfaces of guided gaming chips concurrently. Multiple chips may be exposed to one or more germicidal radiation sources 252 at the same time. Any of a variety of germicidal radiation sources 252 may be used including but not limited to UV-C and/or IR LEDs. Radiation sources 252 may be components of or implemented in part by an LED board assembly 258 included at the interior of housing 210.

Movement of gaming chips 100 may be regulated by one or more chip gates 234 positioned in passages 240. Chip gates 234 can alternate between arresting and releasing positions by use of, for example, an electrical, electronic or mechanical gating device 232. In a further example, gating mechanism 232 is a solenoid or motor. Chip gates 234 may regulate movement of gaming chips 100 by, for example, selectively arresting movement of gaming chips 100 to hold the gaming chips in place while they are being exposed to radiation sources 252. While arrested by chip gates 234, gaming chips 100 may be exposed to radiation from sources 252 directly by line of sight or indirectly by reflection from one or more internal surfaces such as 243 and 244 or by refraction by one or more internal objects. Chip gates 234 may be configured to release a gaming chips 100 to flow downward through passages 240 on to a next chip gate location after a selected amount of time determined based upon, for example, irradiation exposure times sufficient to disinfect the gaming chips.

A position sensor 254 can be used to determine the position of a gaming chip 100 within apparatus 200 for reporting to a user. In some embodiments, apparatus 200 also incorporates a security system 280 configured to restrict access to gaming chips 100 at ingress 220, egress 260, the interior, or a combination of these. Security system 280 can include a mechanical or electronic latch secured by a padlock, keypad lock, smart lock, fingerprint scanner, iris scanner, key card reader, Two-Factor authentication, or a combination of these. Housing 210 can be configured to integrate with security system 280.

In some cases, the interior of the gaming chip disinfection apparatus 200 has one or more valves and or a storage chamber configured to selectively introduce one or more germicidal gases, mists, or vapors into passages 240. In some cases, a single valve can be configured to introduce several phases of germicidal agents.

Controller 290 manages components of apparatus 200 including but not limited to chip gates 234, radiation sources 252, position sensors 254, security system 280 and, in some cases, gas-, mist- or vapor-controlling valves.

FIGS. 1-3 are merely examples, which should not unduly limit the scope of the claims herein. It is to be understood that the specific designation for the apparatus is provided as an example and is not to be construed as limiting the disinfection apparatus to specific numbers, types, or arrangements of the plurality of passages. A person skilled in the art will recognize many variations, alternatives, and modifications of embodiments of the disclosure.

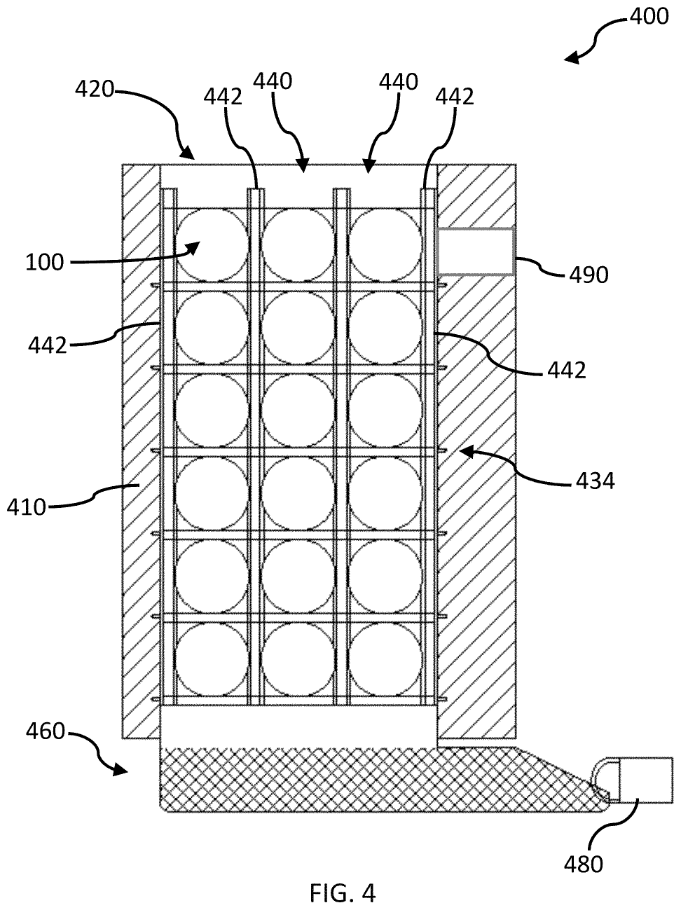

FIGS. 4-6 illustrate another example gaming chip disinfection apparatus 400 configured to disinfect gaming chips 100. Gaming chip disinfection apparatus 400 includes a gaming chip ingress 420, a gaming chip egress 460 and one or more germicidal radiation sources 452 provided to the apparatus interior. Throughout the interior of apparatus 400 gaming chips 100 are guided by channels or passages 440. Housing 410, which may have several exterior walls, surrounds and partially defines the interior.

Referring to FIGS. 4-6, the apparatus can be configured to disinfect multiple gaming chips 100, at once, in a batch-type, semi-batch, or continuous process where multiple chips are exposed one or more germicidal radiation 452 sources at the same time.

Passages 440 are configured to guide gaming chips 100 in parallel (FIG. 4) or along two or more separate paths from ingress 420 to egress 460. Passages 440 can be configured to transport gaming chips 100 from ingress 420 to the interior of apparatus 400 where the chips are disinfected, or irradiated, via radiation sources 452, to an egress 460 that outputs treated disinfected gaming chips to the exterior of the apparatus. Passages 440 are configured to transport gaming chips 100 near germicidal radiation sources 452 such that gaming chips 100 are exposed to incident germicidal radiation for a length of time sufficient to disinfect them.

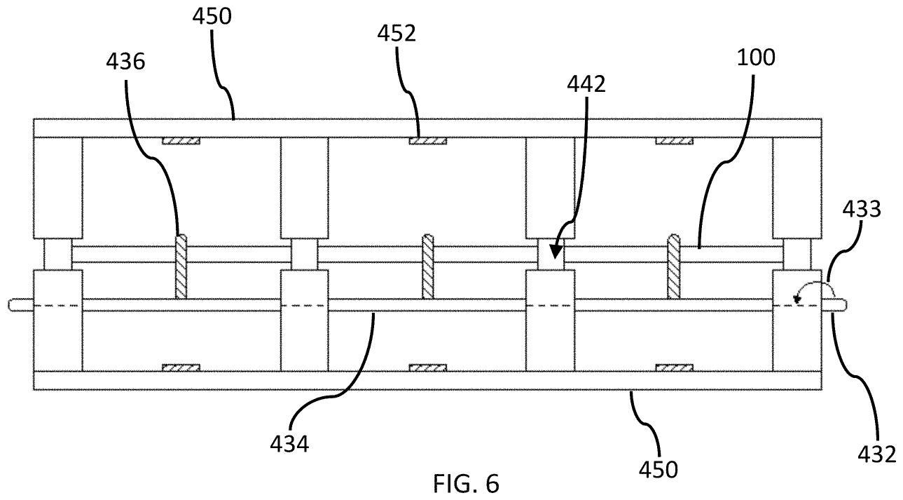

Passages 440 can include opposing walls which parallel one or more of exterior walls of housing 410. The opposing walls can be aligned substantially parallel to top and bottom surfaces of a gaming chip 100, as shown in FIG. 6. In some embodiments germicidal radiation sources 452 are provided to opposing walls of passages 440. In some embodiments, the opposing walls include one or more reflective surfaces. The reflective surfaces can be configured to reflect light, and/or electromagnetic radiation emitted from the radiation sources 452. While the reflective surfaces may be formed from any of a variety of materials, in an example, the reflective services include polished aluminum.

Guiding walls of passages 440 include grooves 442 configured to receive the edge gaming chips 100 and guide gaming chips 100 along passages 440 in an orientation suitable for irradiating at least the top and the bottom base surfaces of the gaming chips substantially concurrently.

Passages 440 may be configured to guide gaming chips from ingress 420, through disinfection apparatus 400 to positions substantially aligned with radiation sources 452 and on to egress 460 under gravity feed. For example, support guiding walls of passages 440 may be vertical.

As with germicidal radiation sources 252, germicidal radiation sources 452 can be element configured to emit photons incident to the surfaces of the gaming chip and capable of disinfecting, or killing germs, such as bacteria, viruses, protozoa, or other microscopic pathogens that can live on the surface of a gaming chip. In some cases, the germicidal radiation source can emit ultraviolet and/or infrared wavelength electromagnetic radiation.

As shown in FIG. 6 germicidal radiation sources 452 may be provided to passages 440 and configured to directly irradiate top and bottom base surfaces of the guided gaming chips concurrently. Any of a variety of germicidal radiation sources 452 may be used including but not limited to UVC and/or IR LEDs. UVC and/or IR LED radiation sources may be components of or implemented in part by an LED board assembly 450 included at the interior of housing 410.

Movement of gaming chips 100 may be regulated by one or more chip gates 436 positioned in passages 440. Chip gates 436 can alternate between arresting and releasing positions by use of, for example, an electrical, electronic or mechanical gating device 432. In a further example, gating mechanism 432 is a solenoid. Chip gates 436 may regulate movement of gaming chips 100 by, for example, selectively arresting movement of gaming chips 100 to hold the gaming chips in place while they are being exposed to radiation sources 452. While arrested by chip gates 436, gaming chips 100 may be exposed to radiation from sources 452 directly by line of sight or indirectly by reflection from one or more internal surfaces or by refraction by one or more internal objects. Chip gates 436 may be configured to release gaming chips 100 to flow downward through passages 440 on to a next chip gate location after a selected amount of time determined based upon, for example, irradiation exposure times sufficient to disinfect the gaming chips.

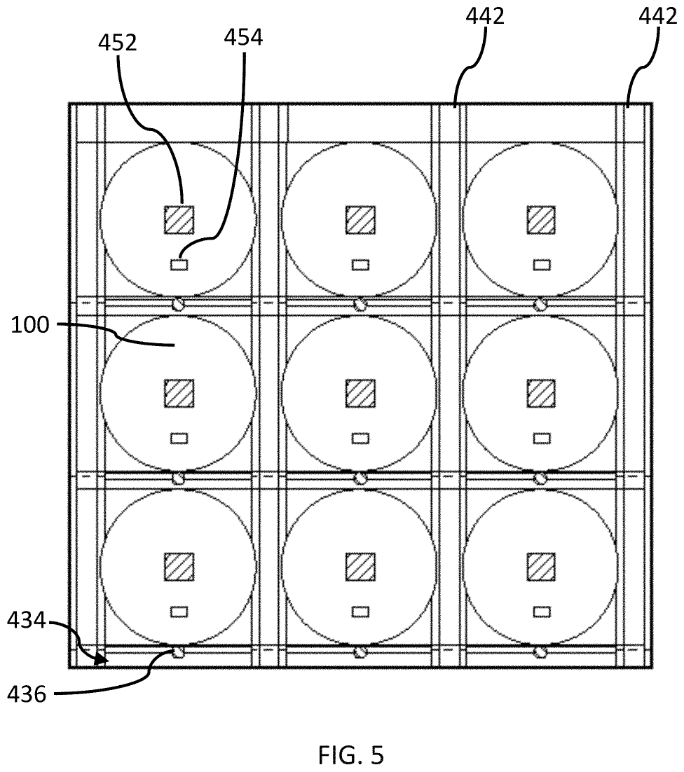

As shown in FIGS. 5 & 6 two or more of chip gates 436 may be configured to concurrently transition between an arresting position and a releasing position. In an example, two or more chip gates 436 are provided to a single shaft 434 which is rotated by gating device 432 about its longitudinal axis in the direction of arrow 433 to open chip gates 436 and allow chips 100 to move to the next chip gate location. Shaft 434 may be rotated about its longitudinal axis in a direction opposite to arrow 433 in order to arrest gaming chips 100 in an irradiation position.

Position sensors 454 can be used to determine the position of a gaming chip 100 within apparatus 400 for reporting to a user. In some embodiments, apparatus 400 also incorporates a security system 480 configured to restrict access to gaming chips 100 at ingress 420, egress 460, the interior, or a combination of these. As with security system 180, security system 480 can include a mechanical or electronic latch secured by a padlock, keypad lock, smart lock, fingerprint scanner, iris scanner, key card reader, Two-Factor authentication, or a combination of these. Housing 410 surrounds the interior of apparatus 400 and can be configured to integrate with security system 480.

In some cases, the interior of the gaming chip disinfection apparatus 400 has one or more valves and or a storage chamber configured to selectively introduce one or more germicidal gases, mists, or vapors into passages 440. In some cases, a single valve can be configured to introduce several phases of germicidal agents.

Controller 490 manages components of apparatus 400 including but not limited to chip gates 436, radiation sources 452, security system 480 and, in some cases, gas- , mist- or vapor-controlling valves.

FIGS. 4-6 are merely examples, which should not unduly limit the scope of the claims herein. It is to be understood that the specific designation for the apparatus is provided as an example and is not to be construed as limiting the disinfection apparatus to specific numbers, types, or arrangements of the plurality of passages. A person skilled in the art will recognize many variations, alternatives, and modifications of embodiments of the disclosure. In another example, a disinfection apparatus may employ a combination series guiding (FIG. 1) and parallel guiding (FIG. 4) arrangements.

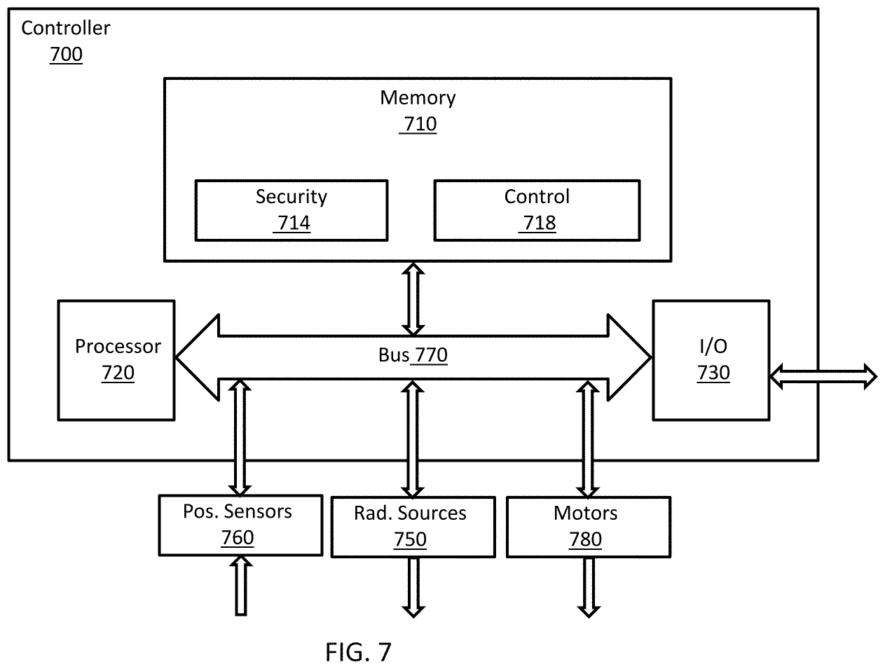

Analog and/or digital control system(s), which may utilize a programmable controller, may be employed to manage, and control various functions of disclosed apparatus. FIG. 7 schematically illustrates an example controller 700 in accordance with an embodiment of the disclosure suitable for performing a variety of control functions for apparatus 100 or 400 including but not limited to alignment of untreated gaming chips 100 at ingress 120 or 420 for entry into passages 140 or 440, conveyance of gaming chips 100 through passages 140, 440, the intensity of germicidal radiation gaming chips 100 are exposed to while in passages 140, 440, or the length of time gaming chips 100 are exposed to germicidal radiation from sources 252 or 452.

Controller 700 may be implemented as control system 190 and/or control system 490 or control system 190 and/or control system 490 may be implemented as controller 700. Controller 700 may include, but is not limited to, a memory 710, a computing hardware such as a processor 720, Input/Output (I/O) devices 730, and a system bus 770 that operatively couples various components including memory 710, the processor 720, and the I/O devices 730.

Controller 700 also includes a power source (not shown) for supplying electrical power to the various components of controller 700. The power source may, for example, include a rechargeable battery, DC or AC power supply or a combination of these.

I/O devices 730 may include a display screen for presenting graphical images to a user of controller 700. In some examples, the display screen may be a touch-sensitive display screen that is operable to receive tactile inputs from the user. These tactile inputs may, for example, include clicking, tapping, pointing, moving, pressing and/or swiping with a finger or a touch-sensitive object like a pen.

Additionally, or alternatively, I/O devices 730 include a mouse or a joystick that is operable to receive inputs corresponding to clicking, pointing, and/or moving a pointer object on the graphical user interface. I/O devices 730 may also include a keyboard that is operable to receive inputs corresponding to pushing certain buttons on the keyboard. Additionally, I/O devices 730 may also include a microphone for receiving an audio input from the user, and a speaker for providing an audio output to the user.

Motors 780 may be implemented as gating devices 232 and/or 432 and vice versa, radiation sources 750 may be implemented as radiation sources 252 and/or 452 and vice versa and position sensors 760 may be implemented as position sensors 254 and/or position sensors 454 and vice versa,

Memory 710 optionally includes non-removable memory, removable memory, or a combination thereof. The non-removable memory, for example, includes Random Access Memory (RAM), Read-Only Memory (ROM), flash memory, or a hard drive. The removable memory, for example, includes flash memory cards, memory sticks, or smart cards.

Memory 710 may store one or more modules configured to cause processor 720 to receive inputs and output control commands. The modules may, for example, be parts of a software product, app or application associated with a chip disinfecting service. In an example a control module 718 is configured to cause processor 720 to activate radiation sources 750 to irradiate gaming chips in a gaming chip disinfection apparatus, to activate solenoids/motors 780 to manipulate chip gates between an arresting position and a releasing position, to interpret data from position sensors 760, and to output information related to the data from position sensors 760 including but not limited to gaming chip position. In an example, security module 714 is configured to cause processor 720 to control access to a gaming chip apparatus ingress, interior, egress or a combination of these.

Further, executing the application on processor 720 may result in generating and rendering a graphical user interface on a display screen. The graphical user interface is configured to facilitate user interactions with a gaming chip disinfection apparatus and/or provide information to the user about gaming chip position, gaming chip disinfection status, gaming chip gate status and/or gaming chip security. Additionally, outputs generated by position sensors 760 may, for example, be indicative of the relative position of gaming chips 100 in passages 140 and/or 440.

When executed on processor 720, control module 718 is configured to resolve and integrate the outputs of the position sensors 760 into useful information about at least one of determining the state of disinfection of the gaming chips 100 accomplished by the length and intensity of germicidal radiation affected onto the gaming chips by the germicidal radiation sources.

In some examples, position sensors 760 may incorporate a timer for including a timestamps with respect to movement of the gaming chips, where the gaming chip through-put of a gaming chip disinfection apparatus can then be determined via output from the position sensors 760. Alternatively, processor 720 may provide system time as reference for including the timestamps with of discrete, treated gaming chips 100, that can be displayed to an operator, demonstrating the treatment of gaming chips 100 once the gaming chips move to an apparatus egress.

Controller 700 is optionally implemented by way of at least one of: a single board microcontroller, a mobile phone, a smart telephone, an MID, a tablet computer, a UMPC, a phablet computer, a PDA and a handheld PC.

FIG. 7 is merely an example, which should not unduly limit the scope of the claims herein. It is to be understood that the specific designation for the control system 700 is provided as an example and is not to be construed as limiting the control system to specific numbers, types, or arrangements of modules and/or components of the system. A person skilled in the art will recognize many variations, alternatives, and modifications of embodiments of the disclosure.

Embodiments of the disclosure provide a gaming chip disinfection apparatus, and method for disinfecting contaminated gaming chips. The gaming chip disinfection apparatus can be utilized for exposing all gaming chip surfaces to a consistent and controlled intensity of germicidal radiation. Additionally, embodiments of the disclosure provide a gaming chip disinfection method enabling disinfection of a gaming chip by passing the chip in a direct line of sight of an ultraviolet or infrared light source.

Disclosed gaming chip disinfecting apparatus may be suitable for use in a method for gaming chip disinfection. An example gaming chip disinfection method includes supplying one or more untreated gaming chips to a gaming chip disinfection apparatus ingress. The gaming chips are guided by several passages from the ingress to one of one or more irradiation positions within the apparatus whereat the gaming chips are arrested by one or more chip gates. While arrested, the top and bottom base surfaces of the gaming chips are concurrently, directly irradiated. The gaming chips are then guided by the several passages from the irradiation positions to one or more additional irradiation positions or to a gaming chip egress. Gaming chips may be guided through the interior of the apparatus in series, in parallel or in a combination of these.

The method may further include preventing unauthorized access to the gaming chips during disinfection. Unauthorized access may be prevented by a security system restricting access to the gaming chips at the ingress, the egress, the apparatus interior, or a combination of these.

In certain embodiments, disinfecting the gaming chips includes selectively arresting movement of gaming chips through the plurality of passages so the gaming chips are directly in line of sight of the radiation sources. The arresting of the gaming chips in line of sight of the radiation sources can be accomplished using one or more chip gates within the plurality of passages.

In an example, selectively arresting movement of gaming chips through the plurality of passages includes translating one or more of the chip gates between an arresting position and a releasing position. The releasing position of the chip gates can be a part of guiding the gaming chips during disinfection utilizing a gravity feed.

In another example, selectively arresting movement of gaming chips through the plurality of passages includes a rotating shaft to rotate the chip gates between an arresting position and a releasing position.

In a further example, disinfecting the gaming chips may further include introducing one or more germicidal gasses into the plurality of passages.

Embodiments of the disclosure are susceptible to being used for various purposes, including, though not limited to, enabling users to disinfect gaming chips.

Modifications to embodiments of the disclosure described in the foregoing are possible without departing from the scope of the disclosure as defined by the accompanying claims. Expressions such as “including”, “comprising”, “incorporating”, “consisting of”, “have”, “is” used to describe and claim disclosed features are intended to be construed in a non-exclusive manner, namely allowing for items, components or elements not explicitly described also to be present. Reference to the singular is also to be construed to relate to the plural.

What is claimed is:

1. A gaming chip disinfection apparatus, comprising:
a gaming chip ingress arranged to control passing of contaminated gaming chips to an interior of the apparatus;
a gaming chip egress arranged to control passing of disinfected gaming chips to an exterior of the apparatus from the interior of the apparatus;
one or more germicidal radiation sources at the interior of the apparatus;
at the interior of the apparatus between the ingress and the egress, a plurality of channels configured to transport gaming chips from the ingress, through the disinfection apparatus and to the gaming chip egress in a controlled manner such that all surfaces of transported gaming chips are simultaneously exposed to at least one of the one or more germicidal radiation sources directly, by line-of-sight for a prescribed duration; and
a security system arranged to restrict access to gaming chips at the ingress, at the egress, at the interior or a combination of these.

2. A gaming chip disinfection apparatus, comprising:
a gaming chip ingress arranged to direct untreated gaming chips to an interior of the apparatus;
a gaming chip egress arranged to direct treated gaming chips to an exterior of the apparatus from the interior of the apparatus;
at the interior of the apparatus, between the ingress and the egress, a plurality of passages arranged to guide gaming chips from the ingress, through the disinfection apparatus and to the gaming chip egress;
a plurality of germicidal radiation sources provided to the passages and configured to irradiate top and bottom base surfaces of the guided gaming chips concurrently; and
a security feature arranged to restrict access to gaming chips at the ingress, at the egress, at the interior or a combination of these.

3. The gaming chip disinfection apparatus as set forth in claim 2, further comprising one or more chip gates provided within each of the plurality of passages and configured to selectively arrest movement of gaming chips through the plurality of passages.

4. The gaming chip disinfection apparatus as set forth in claim 2, wherein the germicidal radiation sources are provided to opposing walls of the plurality of passages.

5. The gaming chip disinfection apparatus as set forth in claim 2, further comprising one or more position sensors provided to each of the plurality of passages.

6. The gaming chip disinfection apparatus as set forth in claim 2, wherein the plurality of passages are configured to guide gaming chips from the ingress, through the disinfection apparatus and to the gaming chip egress under gravity feed.

7. The gaming chip disinfection apparatus as set forth in claim 2, wherein the plurality of passages include one or more incline planes.

8. The gaming chip disinfection apparatus as set forth in claim 2, wherein the plurality of passages include support surfaces with grooves configured to receive sides of gaming chips.

9. The gaming chip disinfection apparatus as set forth in claim 2, wherein the plurality of passages include one or more reflective surfaces.

10. The gaming chip disinfection apparatus as set forth in claim 2, further comprising a valve configured to selectively introduce one or more germicidal gasses into the plurality of passages.

11. A method for gaming chip disinfection, comprising:
supplying one or more untreated gaming chips to a gaming chip disinfection apparatus ingress arranged to control passing of untreated gaming chips to an interior of the apparatus;
with a plurality of germicidal radiation sources of the gaming chip apparatus, directly irradiating top and bottom base surfaces of the guided gaming chips concurrently; and
with a plurality of passages, guiding gaming chips from the ingress, through the disinfection apparatus and to a gaming chip egress.

12. The method as set forth in claim 11, further comprising preventing unauthorized access to the gaming chips during the irradiating.

13. The method as set forth in claim 11, further comprising selectively arresting movement of gaming chips through the plurality of passages with one or more chip gates provided within the plurality of passages.

14. The method as set forth in claim 11, wherein irradiating supplied untreated gaming chips further comprises irradiating with germicidal radiation sources provided to opposing walls of the plurality of passages.

15. The method as set forth in claim 11, further comprising determining position of one or more of the gaming chips within the plurality of passages.

16. The method as set forth in claim 11, wherein guiding gaming chips from the ingress, through the disinfection apparatus and to a gaming chip egress further comprises guiding under gravity feed.

17. The method as set forth in claim 11, wherein guiding gaming chips from the ingress, through the disinfection apparatus and to a gaming chip egress further comprises guiding along one or more incline planes.

18. The method as set forth in claim 11, wherein guiding gaming chips from the ingress, through the disinfection apparatus and to a gaming chip egress further comprises guiding along grooves of support surfaces of the plurality of passages.

19. The method as set forth in claim 11, wherein guiding gaming chips from the ingress, through the disinfection apparatus and to a gaming chip egress further comprises guiding with a plurality of passages including one or more reflective surfaces.

20. The method as set forth in claim 11, further comprising introducing one or more germicidal gasses into the plurality of passages.
